(19) United States
(12) Patent Application Publication
(10) Pub. No.: US 2025/0025587 A1
(43) Pub. Date: Jan. 23, 2025

(54) FLOW REACTOR SYSTEM AND METHOD FOR DISINFECTING A FLUID

(71) Applicant: ams-OSRAM International GmbH, Regensburg (DE)

(72) Inventors: Norbert MAGG, Berlin (DE); Günter HÖTZL, Regensburg (DE); Muhinthan MURUGESU, NORTHVILLE, MI (US)

(21) Appl. No.: 18/574,864

(22) PCT Filed: Jun. 30, 2022
(86) PCT No.: PCT/EP2022/068042

§ 371 Date: Dec. 28, 2023

Related U.S. Application Data
(60) Provisional application No. 63/218,116, filed on Jul. 2, 2021.

(51) Int. Cl. A61L 2/10; A61L 2/00; A61L 2/08; A61L 2/24; A61L 2/26; A61L 9/20; C02F 1/32

(52) U.S. Cl. CPC A61L 2/10; A61L 2/0047; A61L 2/0052; A61L 2/084; A61L 2/24; A61L 2/26; A61L 9/20; C02F 1/325; A61L 2202/11; A61L 2202/14; A61L 2202/22; A61L 2209/111; A61L 2209/12; C02F 2201/005; C02F 2201/3222; C02F 2201/3227; C02F 2201/326; C02F 2209/03; C02F 2209/11; C02F 2209/40; C02F 2301/046

(57) ABSTRACT

A flow reactor system for disinfecting a fluid includes a measuring system for determining at least one state variable, a first irradiation zone and a second irradiation zone in which the fluid is irradiated with electromagnetic radiation. The flow reactor system also includes a first radiation source in the first irradiation zone and a second radiation source in the second irradiation zone. The first radiation source includes at least one emitter unit having a light-emitting diode which emits light in a visible and/or ultraviolet wavelength range. The second radiation source includes at least one emitter unit having a lamp which emits light in an ultraviolet wavelength range. The flow reactor system additionally includes a control unit configured to control the emission intensity of the first radiation source and/or the second radiation source depending on the at least one state variable determined by the measuring system.

In flow-through disinfection plants, a fluid (e.g., air or liquids such as water, milk, blood, etc.) is guided through a reaction zone (e.g., a chamber or a pipe) in which the fluid is exposed to electromagnetic radiation, in particular ultraviolet radiation. For example, this is done linearly, i.e. the fluid enters the reaction zone on one side and leaves it on another side.

LED-based systems (LED: “light emitting diode”) can be used for low flow rates and tight spaces, e.g. for integration in faucets or water dispensers. However, LED-based emitters are comparatively weak in terms of emission intensity, in particular compared to lamp-based systems. LED-based systems may therefore be insufficient to ensure a sufficient level of disinfection at higher flow rates and contamination levels.

For high flow rates, large contamination levels and short exposure times, large UV intensities are required, which can often only be supplied by lamp-based systems. Since UV lamps comprise limited switchability, such systems are usually operated in continuous mode and therefore tend to over-supply UV intensity as the system is configured for the maximum expected values in terms of flow and exposure time.

At least one object of certain embodiments is to specify a flow reactor system for disinfecting a fluid. A further object of certain embodiments is to specify a processing plant comprising a flow reactor system. Furthermore, it is an object of certain embodiments to specify a method for disinfecting a fluid.

These objects are solved by the subject-matter according to the independent patent claims. Advantageous embodiments and further developments of the subject-matter are characterized in the dependent claims and are further apparent from the following description and the drawings.

The present disclosure is based on the idea of providing a flow-through disinfection with optimized power consumption, i.e., a system that allows to provide only as much emission intensity as needed at any given time, and, at the same time, to provide a sufficiently high dose in all conceivable scenarios. To achieve such flexibility, the system uses both LED- and lamp-based emitters.

Here and in the following, “radiation” or “light” may in particular refer to electromagnetic radiation having one or more wavelengths or wavelength ranges. In particular, light or radiation described here and in the following may be ultraviolet light or visible light and may comprise or be wavelengths or wavelength ranges from the UV-C (280-100 nm), UV-B (315-280 nm), UV-A (380-315 nm), and/or visible (380-780 nm) spectral ranges.

According to at least one embodiment, a flow reactor system is provided for disinfecting a fluid. The flow reactor system comprises a measuring system for determining at least one state variable. Further, the flow reactor system comprises a first irradiation zone and a second irradiation zone in which the fluid is irradiated with electromagnetic radiation. The flow reactor system further comprises a first radiation source arranged in the first irradiation zone. The first radiation source comprises at least one emitter unit comprising a light emitting diode, which emits light in a visible and/or ultraviolet wavelength range during operation. The flow reactor system further comprises a second radiation source arranged in the second irradiation zone. The second radiation source comprises at least one emitter unit comprising a lamp, which emits light in an ultraviolet wavelength range during operation. Furthermore, the flow reactor system comprises a control unit. The control unit is provided and configured to control the emission intensity of the first radiation source and/or the second radiation source depending on the at least one state variable determined by the measuring system.

The term fluid comprises both gases and liquids. This means that the fluid can be both a gaseous and a liquid medium. For example, the fluid is air or another gas. The fluid can also be water, milk, blood, or any other liquid.

Disinfection of the fluid can mean, in particular, that microorganisms such as viruses, bacteria and fungi in the fluid are at least reduced. To ensure a sufficient degree of sterilization of the fluid, for example, a residual content of microorganisms capable of reproduction in the fluid after disinfection can be less than 10⁻²% (log 4 reduction), less than 10⁻³% (log 5 reduction, less than 10⁻⁴% (log 6 reduction), less than 10⁻⁵% (log 7 reduction), or even smaller residual contents.

The measuring system may comprise one detector system or multiple detector systems, wherein each detector system contributes to the determination of at least one state variable. For example, the measuring system comprises a detector system for determining a flow rate of the fluid. For this purpose, the measuring system may comprise a sensor which is configured as a flow rate sensor to measure a flow rate of the fluid and output it as a state variable. Alternatively or additionally, the measuring system comprises a detector system for determining a pressure of the fluid.

Alternatively or additionally, the measuring system comprises a detector system for determining a turbidity level of the fluid. A measurement of turbidity can be based, for example, on a measurement of light scattering: one or more emitters that emit light in, for example, a visible (VIS), infrared (IR), or ultraviolet (UV) wavelength range are arranged in a predeterminable orientation relative to corresponding light sensors, wherein the fluid is located between the light emitter and the light sensor. If a transmitted radiation component is measured, the signal received by the light sensors decreases due to light scattering with increasing turbidity of the fluid. On the other hand, if a scattered radiation component is measured, the signal received by the light sensors due to light scattering increases with increasing turbidity of the fluid. The turbidity level can affect the penetration depth of UV radiation into the fluid. At high turbidity levels, a higher emission intensity of the radiation sources may be necessary to ensure sufficient sterilization of the fluid.

Alternatively or additionally, the measuring system comprises a detector system for determining a contamination level of the fluid. A measurement of the contamination level can be performed, for example, by exciting a characteristic fluorescence of the microorganisms in the fluid: For example, a UV light source may be arranged in a predeterminable orientation relative to corresponding fluorescence sensors, wherein the fluid is located between the UV light source and the fluorescence sensor. By the irradiation with UV light, a characteristic fluorescence of the microorganisms contained in the fluid is excited. The fluorescence sensor can measure the fluorescence signal emitted by the microorganisms. The fluorescence sensor may comprise, for example, a photodiode and a filter, wherein the filter is provided and configured to block out radiation in non-relevant spectral ranges, in particular the excitation radiation wavelength present. The UV light source of the detector system may be a UV light source dedicated for the measuring system. Alternatively, the UV light source of the detector system may be formed by the first or second radiation source. This means that at least parts of the first or second radiation source can also serve as UV light source for the excitation of a characteristic fluorescence of the microorganisms in the fluid. Advantageously, a dedicated UV light source can thus be omitted.

The measuring system can comprise further detector systems/sensors to determine further state variables. The above-described examples are not exhaustive. For example, the measuring system may comprise a detector system to determine an “external” state variable, i.e., a state variable independent of the fluid.

The first radiation source may comprise one or more emitter units, wherein each emitter unit comprises at least one LED. In particular, the first radiation source comprises a plurality of emitter units which are preferably arranged in one or more arrays. For example, the first radiation source comprises a plurality of LED arrays. LEDs have some advantages compared to lamps, for example small dimensions in the range from about 1 mm² to about 1 cm². Due to their small size, a high flexibility can be achieved, so that the radiation source can be adapted to the system. The small size also makes it possible to arrange individual LEDs in arrays and pixels. Furthermore, LEDs have a low operating heat and allow fast switching cycles. The emitted wavelength of LEDs can be specifically adapted and optimized with regard to the absorption properties of application-typical microorganisms such as viruses, bacteria, and fungi. For example, LEDs can be configured to emit radiation in the 260-280 nm range. LEDs exhibit high mechanical stability, have a long lifetime, and can be manufactured mercury-free. The emitted light intensity of LEDs can be adjusted within a range of approximately 1-100% of the nominal power by varying the drive current. However, LED-based UV radiation sources are rather weak in terms of UV intensity, wherein an output power can be in the range of 5-200 mW per LED.

The second radiation source may comprise one or more emitter units, wherein each emitter unit comprises at least one lamp. The lamp does not comprise an LED as an illuminant. This means that the second radiation source is free of an LED. For example, the lamp is a discharge lamp. In particular, the lamp may be a low-pressure discharge lamp. Medium-pressure or a high-pressure discharge lamp are also possible. In particular, the lamp may contain mercury and/or amalgam. Compared to LEDs, UV lamps are available in a wide power range from about 5 W to several kW per lamp. Furthermore, the cost with regard to output power ($/W) can be lower than for LEDS.

In the irradiation zones, the fluid is exposed to electromagnetic radiation, in particular UV radiation. Visible light can also contribute to a disinfection of the fluid. For example, a multifrequency spectrum can cause irreversible damage to the structure of exposed microorganisms (e.g., viruses such as SARS-COV-2) and kill a large proportion of microorganisms.

Depending on the time variations of one or more state variables, a higher or lower irradiation dose is required for sufficient disinfection. To ensure a sufficient degree of sterilization, the radiation dose to which the contaminated fluid is exposed must be large enough. According to the formula

$$dose ( mJ / {cm}^{2} ) = intensity ( mW / {cm}^{2} ) \times exposure time ( s ) ,$$

the dose depends on the intensity emitted by the radiation sources and the time the fluid is exposed to this intensity. Consequently, higher intensities require smaller exposure times and vice versa. Certain applications require clear demands for the throughput or the flow rate of the system, e.g., in terms of the volume of fluid that must pass through the system per unit of time. Therefore, there may not be much latitude in the design of a flow reactor system in terms of exposure time. Accordingly, the flow reactor system is configured in such a way that the intensity, i.e. the emission intensity of the radiation sources, can be adapted to the present situation.

For this purpose, the control unit is configured to control the emission intensity of the first radiation source and/or the second radiation source.

The control unit is connected to the measuring system. The control unit receives the determined state variable or several state variables from the measuring system. Alternatively, the control unit determines the state variable or several state variables from the measurement data measured by the measuring system. The control unit controls the emission intensity of the radiation sources depending on the at least one state variable. This may mean, for example, that the emission intensity is increased if a threshold value of the state variable is exceeded. The emission intensity may be reduced if a threshold value of the state variable is undershot. The control can be carried out in steps or stepless, i.e. continuously.

The control unit is further connected to the first radiation source and to the second radiation source. The control unit may be connected to the first radiation source via a driver unit of the first radiation source. For example, the control unit may vary a drive current for operating the first radiation source. For example, the control unit may vary the emission intensity of the first radiation source by switching on, dimming, or switching off at least one emitter unit of the first radiation source.

The control unit may be connected to the second radiation source via a driver unit of the second radiation source. For example, the control unit may vary a drive current for operating the second radiation source. For example, the control unit may vary the emission intensity of the second radiation source by switching on, dimming, or switching off at least one emitter unit of the second radiation source.

Advantageously, the flow reactor system comprises both LEDs and lamps as radiation sources. Thus, it is possible to use the above-mentioned advantages of both types of radiation sources. In particular, flow-through disinfection can be provided with optimized power consumption, i.e. the emission intensity can be varied by a control unit depending on the scenario and on one or more state variables. In this context, the lamp-based second radiation source can be used in particular to disinfect the fluid at, for example, high contamination levels, high flow rates and/or high turbidity levels. The LED-based first radiation source, on the other hand, can be used to disinfect fluids with, for example, low levels of contamination and/or to respond to small fluctuations in one or more state variables. In this way, the flexibility of the flow reactor system can be increased and its energy consumption can be optimized.

According to at least one further embodiment, in the flow reactor system, the measuring system, the first irradiation zone and the second irradiation zone are arranged along a flow channel of the fluid.

Alternatively, the measuring system is not arranged at the flow channel. The measuring system can be locally separated from the irradiation zones and the flow channel. This can be the case, in particular, if the measuring system is provided and configured to determine an “external” state variable, i.e., a state variable that is not or only indirectly connected to the fluid and/or the flow reactor system. For example, the flow reactor system may be integrated in a room ventilation system or air conditioning system connected to a room and intended to disinfect the air in the room. In this case, for example, the measuring system may be configured to measure an air quality (CO₂ content or contamination) in a room and output it as a state variable. This measurement can be performed at a suitable location in the room ventilation system or air conditioning system. Alternatively, the measurement can be performed at one or more locations in the room. In this case, the measuring system can also be configured to determine a “qualitative” state variable, such as the number of people in the room, and output it as a state variable. The contamination level of the air in the room depends only indirectly on the number of people in the room. For example, by counting the number of people entering or leaving a room through a light barrier, it can be determined how many people are in the room. As an alternative to light barriers, camera systems or infrared sensor systems such as thermal imaging cameras can also be used.

At least one section of the flow channel can be configured as a first irradiation zone. At least one further section of the flow channel may be formed as a second irradiation zone. The flow reactor system may also comprise more than one first irradiation zone. The flow reactor system may also comprise more than one second irradiation zone. For example, first and second irradiation zones may alternate along the flow channel. The flow channel may be formed as a pipe or pipe system. This may mean that the fluid to be disinfected flows through the pipe or pipe system. For example, the pipe or pipe system may comprise polytetrafluoroethylene (PTFE) as a material. Other materials for the pipe or pipe system are also possible.

The first and/or the second radiation source can be arranged in and/or at the flow channel. Emitter units of the first and second radiation source can couple radiation into the flow channel from different directions. For example, the flow channel is surrounded by emitter units in lateral directions, wherein lateral directions are perpendicular to the flow direction of the fluid. Emitter units of the first and second radiation source can be arranged outside the flow channel and couple light, in particular UV radiation, into the flow channel via transparent windows. For example, UV-transparent windows made of quartz glass can be integrated in a flow channel configured as a pipe, via which UV radiation can be coupled into the flow channel. Emitter units of the second radiation source and in particular of the LED-based first radiation source can also be arranged in the flow channel. For example, the flow channel may comprise chambers in which the emitter units are inserted. Boundaries of the chambers may be transparent to couple in the electromagnetic radiation. Here and in the following, “transparent” refers to a transparency for electromagnetic radiation of at least 80% or at least 90%.

Advantageously, the flow reactor system may be compact. For example, the measuring system, the first irradiation zone and the second irradiation zone can be arranged along a short pipe section and the radiation sources can be mounted in or on the pipe.

According to at least one further embodiment, the second irradiation zone is arranged after the first irradiation zone in the flow direction of the fluid. A distance between the first irradiation zone and the second irradiation zone can be greater than a distance between the measuring system and the first irradiation zone. Switching times of the LED-based first radiation source are short, whereas switching times of the lamp-based second radiation source are comparatively long (for example, low-pressure mercury vapor discharge lamps comprise a ramp-up time of several minutes). Ideally, therefore, it is possible to react quickly to changes in the state variable determined by the measuring system by adjusting the emission intensity of the first radiation source. For the second radiation source arranged in the second radiation zone positioned behind, there is more time for an adjustment of the emission intensity. The advantageous arrangement of the first and second irradiation zones takes into account the switching times of the first and second radiation sources.

According to at least one further embodiment, the first irradiation zone and the second irradiation zone are arranged after the measuring system in the flow direction of the fluid.

This means that changes in the state variable determined by the measuring system can ideally be responded to quickly by adjusting the emission intensity of the first and second radiation sources. If the measuring system is arranged at the flow channel, a distance of the measuring system to the first irradiation zone can be small because the switching times of the LED-based first radiation source are short. In particular, the distance of the measuring system to the first irradiation zone can be smaller than a distance between the first irradiation zone and the second irradiation zone.

In an alternative embodiment, the first irradiation zone and/or the second irradiation zone are located before the measuring system in the flow direction of the fluid. This can be the case, in particular, if the flow reactor system comprises a recirculation channel. In a further embodiment, the measuring system is arranged between the first and second irradiation zones. A measuring system arranged after the irradiation zone can determine at least one state variable after UV irradiation, which can provide information about the success of the irradiation with regard to disinfection of the fluid.

According to at least one further embodiment, the state variable determined by the measuring system comprises at least one of the following group: a contamination level of the fluid, a flow rate of the fluid, a turbidity level of the fluid, a pressure of the fluid, a state variable independent of the fluid.

This can mean that the measuring system determines a contamination level of the fluid and outputs it as a state variable. Alternatively or additionally, the measuring system determines a flow rate of the fluid and outputs the determined value as a (further) state variable. Alternatively or additionally, the measuring system determines a turbidity level of the fluid and outputs the determined value as a (further) state variable. Alternatively or additionally, the measuring system determines a pressure of the fluid and outputs the determined value as a (further) state variable. Alternatively or additionally, the measuring system determines a state variable independent of the fluid, as exemplified above.

In at least one embodiment, a combined state variable is formed from a plurality of the measured values determined by the measuring system. For example, both the contamination level of the fluid and the flow rate are taken into account in the combined state variable. The measuring system comprises suitable detector systems with corresponding sensors for determining the respective state variables, as explained above. Advantageously, a plurality of state variables can be determined by the measuring system in order to control the emission intensity of the radiation sources in dependence thereon.

According to at least one further embodiment, the flow reactor system comprises a further detector system for detecting the radiation emitted by the radiation sources. The determined measured value can serve as a reference value. For example, the nominal power of the radiation sources can thus be monitored during operation or a degradation of the radiation sources dependent on the operating time can be compensated.

According to at least one further embodiment, the flow reactor system comprises a recirculation channel. The recirculation channel is provided and configured to recirculate a portion of the fluid at a position which is located after the first and/or second irradiation zone to a position before the irradiation zones.

Like the flow channel, the recirculation channel can be configured as a pipe or pipe system. Whether or not a portion of the fluid is recirculated via the recirculation channel to a position before the irradiation zones can depend on one or more state variables. For example, if the measuring system determines that the fluid comprises a high contamination level, at least a portion of the fluid may be recirculated to be subjected to a repeated irradiation in the irradiation zones. A flow reactor system that comprises a recirculation channel is able to respond to large variations in a state variable and provide adequate disinfection of the fluid.

According to at least one further embodiment, the flow reactor system further comprises valves for opening and closing an inlet and an outlet of the recirculation channel.

This can mean that at least one valve is provided and configured to open or close an inlet of the recirculation channel. At least one further valve is provided and configured to open or close an outlet of the recirculation channel. The valves may be configured as two-way valves. The valves may be configured to control whether and/or how much fluid is recirculated via the recirculation channel to a position before the irradiation zones. The valves can be connected to and controlled by the control unit.

The access of the recirculation channel represents a branch of the flow channel, at which the fluid can flow from the flow channel into the recirculation channel. The inlet of the recirculation channel is arranged after at least one irradiation zone in the flow direction. The outlet of the recirculation channel represents a branch of the flow channel, at which the fluid can flow from the recirculation channel into the flow channel. The outlet of the recirculation channel is arranged before at least one irradiation zone in the flow direction. Advantageously, the valves can be used to control whether and/or how much fluid is recirculated to a position before the irradiation zones.

According to at least one further embodiment, the recirculation channel comprises a buffer region. The buffer region is provided and configured to receive a portion of the fluid. The buffer region can be configured as a container. If the recirculation channel is configured as a pipe, the buffer region can be configured, for example, as a widening of the pipe.

The use of a buffer region that can receive a portion of the fluid can be advantageous, for example, in order to retain at least a portion of the fluid in the event of sudden and/or unusually large fluctuations in one of the state variables and expose it to a subsequent repeated irradiation. In this way, a sufficient degree of fluid sterilization can be ensured. A flow reactor system that comprises a recirculation channel with a buffer region is capable of balancing fluctuation peaks in one of the state variables. For example, the recirculation channel/buffer region is needed only in exceptional cases, i.e., not by default, which allows the flow rate to be kept high. The recirculation channel/buffer region can be a safeguard for the flow reactor system to be able to guarantee a desired disinfection rate by accepting a drop in the flow rate.

According to at least one further embodiment, the emitter units of the first radiation source and the second radiation source emit light in the UV-C wavelength range during operation. For example, during operation, the emitter units may emit light in different wavelength ranges within the UV-C range. For example, a subset of the emitter units, in particular the lamp-based emitter units of the second radiation source, may emit light at 254 nm, while another subset of the emitter units, in particular the LED-based emitter units of the first radiation source, may emit light in the 265-280 nm range. The emitter units of the first and second radiation source may form a plurality of subsets, each emitting light in different wavelength ranges, in particular within the UV-C wavelength range.

The UV-C spectral range is well suited in terms of UV absorption characteristics to reduce application-typical microorganisms such as viruses, bacteria and/or fungi and to disinfect the fluid. Advantageously, the emitter units cover a wide range of wavelengths. In particular, by combining two or more UV-C wavelength ranges, the “appropriate” wavelength for different microorganisms can be provided, i.e. the wavelength with maximum UV absorption for the corresponding microorganisms.

According to at least one further embodiment, the first radiation source comprises at least one further emitter unit comprising a light-emitting diode. The further emitter unit emits light in the UV-A wavelength range during operation. Alternatively or additionally, the further emitter unit emits light in the visible wavelength range.

This means that constant UV-A radiation between 315-380 nm can additionally be applied to irradiate the fluid with it. These wavelengths can be provided by LED light sources with comparatively high output power and at low cost. It has been shown in studies that irradiation with light of these wavelengths can contribute to disinfection, for example with healthcare-associated infections (HAI).

Alternatively or additionally, the at least one additional emitter unit emits light in the visible wavelength range during operation, i.e. in the range 380-780 nm, in particular in the violet wavelength range (380-430 nm). Multiple additional emitter units can emit light in different wavelength ranges during operation, making it possible to irradiate the fluid with a combination of wavelength ranges or frequencies, wherein the irradiation with light of each wavelength range contributing to disinfection of the fluid.

According to at least one further embodiment, the flow reactor system also comprises at least one further measuring system arranged after the measuring system in the flow direction of the fluid. The further measuring system is provided and configured to redetermine the at least one state variable determined by the measuring system.

The further measuring system can determine the state variable determined by the measuring system at a different time and/or at a different position of the flow channel. The measuring system can be arranged before the irradiation zones in the flow direction of the fluid, whereas the further measuring system can be arranged after at least one irradiation zone in the flow direction of the fluid. In this way, the state variable determined by the measuring system can be redetermined after irradiation of the fluid in order to be able to draw conclusions about the success of the irradiation. If it is determined via the redetermination of the state variable that the disinfection of the fluid was not yet sufficient, the fluid can, for example, be recirculated via the recirculation channel to a position before the irradiation zones or the emission intensity of the radiation source can be increased in a subsequent irradiation zone.

It is also possible that both the measuring system and the further measuring system are arranged before the irradiation zones. The measuring system and the further measuring system can be spaced apart from each other. Due to the redundant determination of at least one state variable, a better prediction of “high-level events” can be achieved, i.e. events that require a high radiation intensity of the radiation sources. According to at least one embodiment, the further measuring system determines the same and/or a different state variable than the state variable determined by the measuring system.

According to at least one further embodiment, the further measuring system is provided and configured to determine a further state variable different from the state variable determined by the measuring system. For example, the measuring system measures a turbidity level or a fluid pressure, whereas the further measuring system measures a contamination level of the fluid.

According to at least one further embodiment, the flow reactor system further comprises an analysis system. The analysis system is provided and configured to determine patterns in a temporal and/or spatial progression of the at least one state variable and to provide pattern information to the control unit.

The analysis system can be a computer on which a suitable software is executed. For example, the software can be AI-based (AI: “artificial intelligence”) or use methods based on deep learning or machine learning. The analysis system can be part of the control unit or separated from it. The analysis system is connected to the measuring system and/or to the control unit. The analysis system analyzes the measured values or state variables determined by the measuring system. In this way, it is possible to determine eventual patterns in a temporal and/or spatial progression of the at least one state variable. The patterns can be typical for the application. For example, the analysis system determines that an increased contamination level of the fluid occurs every day at a comparable time of day. The analysis system provides information on possible patterns to the control unit. The analysis system may additionally communicate further information to the control unit. For example, based on the pattern information, the analysis system communicates control data to the control unit as to when and in what manner the control unit should control the emission intensity of the radiation sources. The control unit can respond to the information and/or control data from the analysis system by varying the emission intensity of the radiation sources.

According to at least one further embodiment, at least one emitter unit of the first radiation source emits light in a direction that is not perpendicular to the flow direction of the fluid. This may mean that a main radiation direction of the emitter unit is not perpendicular to the flow direction of the fluid. For example, the emitter unit couples radiation into the flow channel at an angle between 30° and 60°. If the emitter unit is located in the flow channel, the emitter unit can also emit radiation in the flow direction (0°) or against the flow direction (180°). By emitting light at angles that are not perpendicular to the flow direction of the fluid, the fluid is exposed to UV radiation for a longer period of time. This can increase the irradiation time compared to perpendicular irradiation.

In a further exemplary embodiment, the emitter unit emits light perpendicular to the flow direction. In a further exemplary embodiment, light is reflected at boundaries of the flow channel, e.g. on the pipe, at a variety of angles so that the light is still available for disinfecting the fluid after reflection. In this way, the radiation is used optimally.

According to at least one further embodiment, a processing plant comprises such a flow reactor system. This means that all features disclosed for the flow reactor system are also disclosed for the processing plant and vice versa. The flow reactor system may be integrated into the processing plant. In addition to the flow reactor system, additional purification and/or filtration systems may be implemented in the processing plant. For example, the processing plant may be a processing plant for liquids, such as water. That is, the processing plant may be a water processing plant. The processing plant may also be a processing plant for gases, for example for air. In this case, the processing plant may be, for example, an air conditioning unit or an air purification unit.

According to at least one further embodiment, a method for disinfecting a fluid in a flow reactor system is specified. All features disclosed for the flow reactor system are also disclosed for the method and vice versa.

According to the method, at least one state variable is determined by a measuring system. Furthermore, the method comprises the guidance of the fluid through a first irradiation zone and through a second irradiation zone, in which the fluid is irradiated with electromagnetic radiation. In the first irradiation zone, a first radiation source is arranged which comprises at least one emitter unit comprising a light emitting diode. During operation, the emitter unit emits light in a visible and/or ultraviolet wavelength range.

A second radiation source is arranged in the second irradiation zone, which comprises at least one emitter unit comprising a lamp. During operation, the emitter unit emits light in an ultraviolet wavelength range. Furthermore, the method comprises the control of the emission intensity of the first radiation source and/or the second radiation source. The control of the emission intensity is dependent on the at least one state variable determined by the measuring system.

According to the method, both LEDs and lamps are advantageously used as radiation sources for disinfection of the fluid. A flow-through disinfection with optimized power consumption can be provided, i.e. the emission intensity can be varied by a control unit depending on the scenario and on one or more state variables. In this context, the lamp-based second radiation source can be used in particular to disinfect the fluid at, for example, high contamination levels, high flow rates and/or high turbidity levels. The LED-based first radiation source, on the other hand, can be used to disinfect fluids with, for example, low contamination levels and/or to respond to small fluctuations in one or more state variables. In this way, energy consumption can also be optimized.

According to at least one further embodiment, the control of the emission intensity comprises dimming or switching off at least one emitter unit of the first radiation source if a first threshold value of the at least one state variable is undershot.

The first threshold value of the at least one state variable can, for example, mark a transition of the contamination level, the flow rate or the turbidity level of the fluid etc. from a lower to a higher value. Multiple threshold values may also be defined to realize a multi-level control. Alternatively, the control of the emission intensity can be realized stepless, i.e. the emission intensity of the first radiation source can be varied continuously depending on the value of the state variable, for example according to a linear function between state variable and emission intensity. In this case, the first threshold value is a value in a continuous spectrum of values of the state variable.

For example, during times when there is no flow of the fluid or no contamination of the fluid, all emitter units of the first (and second) radiation source can be turned off. During times of low flow, low contamination and/or low turbidity of the fluid, only the emitter units in the first irradiation zone need to be switched on. The first irradiation source is LED-based. Since the LED circuit is fast, the distance, depending on the flow rate and the time needed for the analysis, between the measuring system and the first irradiation zone can be small. At very low contamination levels, flow rates, and/or turbidity levels, the LEDs can be dimmed and/or only a portion of the LEDs in an LED array can be switched on. At higher contamination levels, flow rates, and/or turbidity levels, all LEDs can be turned on at full nominal power, if necessary. For example, the emission intensity of the first radiation source can be controlled via a control unit.

According to at least one further embodiment, the control of the emission intensity comprises dimming or switching off of at least one emitter unit of the second radiation source, if a second threshold value of the at least one state variable is undershot.

The second threshold value is different from the first threshold value. The second threshold value of the at least one state variable can, for example, mark a further transition of the contamination level, the flow rate or the turbidity level of the fluid etc. from a lower to a higher value. Multiple threshold values may also be defined to realize a multi-level control. Alternatively, the control of the emission intensity can be realized stepless, i.e. the emission intensity of the second radiation source can be varied continuously depending on the value of the state variable, for example according to a linear function between state variable and emission intensity. In this case, the second threshold value is a value in a continuous spectrum of values of the state variable.

In the case that one or more state variables exceed a maximum threshold for LED-only irradiation, one or more of the lamp-based emitter units of the second radiation source in the second irradiation zone can be additionally switched on. If the state variables fall below the maximum threshold for LED-only lighting again, the lamp-based emitter units of the second radiation source can be switched off again. For a slow decrease of the state variable, the emitter units of the second radiation source can be dimmed gradually or continuously or switched off individually. For example, the emission intensity of the second radiation source can be controlled by a control unit.

According to at least one further embodiment, the method comprises recirculating a portion of the fluid at a position after the first and/or second irradiation zones to a position before the irradiation zones if a third threshold value of the at least one state variable is exceeded.

The third threshold value is different from the first and second threshold value. The third threshold value of the at least one state variable can, for example, mark a further transition of the contamination level, the flow rate or the turbidity level of the fluid etc. from a lower to a higher value. Multiple threshold values may also be defined to realize a multi-stage control. Alternatively, the control of the fluid recirculation can be realized stepless, i.e. the amount of recirculated fluid fraction can be varied continuously depending on the value of the state variable, for example according to a linear function between state variable and recirculation amount. In this case, the third threshold value is a value in a continuous spectrum of values of the state variable.

The recirculation of a portion of the fluid can take place via a recirculation channel, which connects a position of the flow channel after the first and/or second irradiation zone with a position of the flow channel before the irradiation zones. Valves may be arranged at an inlet and an outlet of the recirculation channel. For example, the valves can be regulated by a control unit to control whether and/or how much fluid is recirculated to be irradiated with UV light a further time in the irradiation zones.

According to at least one further embodiment, the control of the emission intensity comprises the activation of the second radiation source if a warning level of the at least one state variable is exceeded. The second radiation source is deactivated if the warning level or a switch-off level of the state variable is undershot.

In the event that one or more of the state variables exceeds a maximum threshold level for LED-only illumination, one or more of the lamp-based emitter units of the second radiation source are switched on. Ideally, the activation of the second radiation source already starts if at least one of the state variables exceeds the warning level, which is significantly below the maximum threshold for LED-only irradiation, because the lamp-based emitter units of the second radiation source may comprise a long ramp-up phase. The switch-off level to be undershot for the deactivation of the second radiation source no be a different level than the warning level to be exceeded for the activation of the second radiation source in order to achieve a hysteresis effect.

Further embodiments of the method for disinfecting a fluid will be apparent to the skilled reader from the embodiments of the flow reactor system described above.

The foregoing and following descriptions apply equally to the flow reactor system, the processing plant including the flow reactor system, and the method in the flow reactor system for disinfecting a fluid.

Further advantages, advantageous embodiments and developments result from the exemplary embodiments described below in connection with the figures.

In the exemplary embodiments and figures, the same, similar or similar-acting elements may each be provided with the same reference signs. The elements shown and their proportions to one another are not to be regarded as true to scale; rather, individual elements, such as layers, components, devices, and areas, may be shown exaggeratedly large for better representability and/or for better understanding.

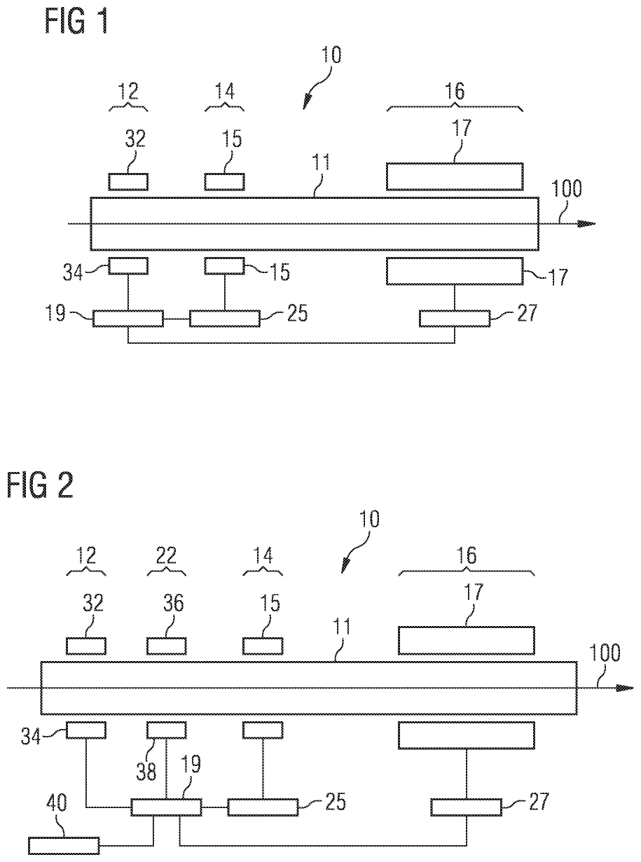

FIG. 1 shows a schematic illustration of a flow reactor system according to an exemplary embodiment.

FIG. 2 shows a schematic illustration of a flow reactor system according to further exemplary embodiments.

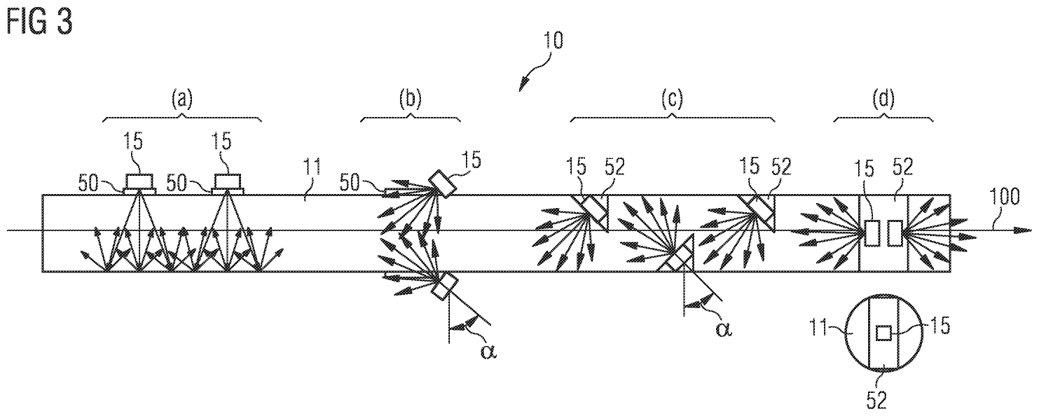

FIG. 3 shows schematic illustrations of various arrangement options for emitter units according to further exemplary embodiments.

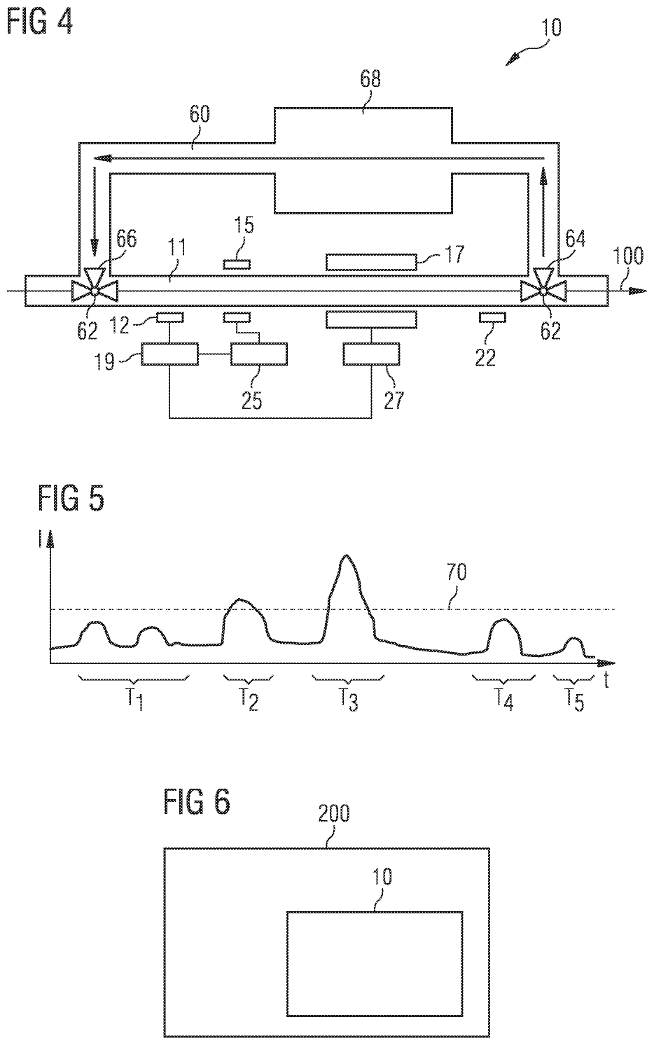

FIG. 4 shows a schematic illustration of a flow reactor system according to further exemplary embodiments.

FIG. 5 shows a schematic illustration of a method for varying the emission intensity over time according to an exemplary embodiment.

FIG. 6 shows a schematic illustration of a processing plant with a flow reactor system according to an exemplary embodiment.

In connection with FIG. 1, an exemplary embodiment for a flow reactor system 10 is shown, by means of which a possible arrangement concept is to be illustrated.

The flow reactor system 10 is provided to disinfect a fluid. The flow reactor system 10 comprises a flow channel 11 in which a fluid flows along a flow direction 100. The flow direction 100 is illustrated with an arrow. The fluid may be a liquid or a gas, such as air, water, milk, blood, etc. The flow channel 11 may, for example, be formed as a pipe or pipe system such that the fluid is completely contained within the flow channel 11. This may mean that the fluid is completely enclosed by the pipe or pipe system. The flow channel 11 formed by the pipe may be part of a larger pipe system. The flow channel 11 may be rectilinear as shown, but a curved flow channel 11 is also possible.

The flow reactor system 10 comprises a measuring system 12 for determining at least one state variable. In the example shown, the measuring system 12 is located at an inlet of the flow channel 11, i.e., at an inlet-side position of the flow channel 11. As shown, the measuring system 12 may comprise multiple components. In the example shown, the measuring system 12 comprises a light emitter 32 and a light sensor 34. The light emitter 32 and the light sensor 34 may form a detector system of the measuring system 12 to detect one or more state variables. For example, the detector system is configured to measure a contamination level, a flow rate, a turbidity level, or a pressure of the fluid. The respective measurement result may be output as a state variable. The measuring system 12 may comprise further detector systems/sensors for determining further state variables.

The flow reactor system 10 according to FIG. 1 further comprises a first irradiation zone 14 and a second irradiation zone 16, in which the fluid is irradiated with electromagnetic radiation. In the exemplary embodiment according to FIG. 1, the measuring system 12, the first irradiation zone 14 and the second irradiation zone are arranged along the flow channel 11. The first irradiation zone 14 is arranged between the measuring system 12 and the second irradiation zone 16. In the example shown, the second irradiation zone 16 is located at an outlet of the flow channel 11, i.e., at an outlet-side position of the flow channel 11. A distance between the measuring system 12 and the first irradiation zone 14 may be small, in particular smaller than a distance between the first irradiation zone 14 and the second irradiation zone 16.

A first radiation source 15 is arranged in the first irradiation zone 14. The first radiation source 15 comprises at least one emitter unit comprising a light emitting diode (LED). During operation, the first radiation source 15, i.e. the at least one LED-based emitter unit, emits light around visible (VIS) and/or ultraviolet (UV) wavelength range. In particular, the first radiation source 15 emits light in the UV-C spectral range, for example in the range between 260 nm and 270 nm. The first radiation source 15 may comprise a plurality of emitter units, each emitter unit comprising a LED. The first radiation source 15 may comprise only LEDs. In particular, the LED-based emitter units of the first radiation source 15 may be arranged in one or more arrays. The emitter units of the first radiation source 15 may irradiate the fluid with light from different directions. For example, and as indicated in FIG. 1, the emitter units of the first radiation source 15 may be arranged annularly around the flow channel 11.

A second radiation source 17 is arranged in the second irradiation zone 16. The second radiation source 17 comprises at least one emitter unit comprising a lamp. The lamp may, for example, be a discharge lamp. In particular, the second radiation source 17 may be free of a LED. During operation, the second radiation source 17, i.e. the at least one lamp-based emitter unit, emits light around UV wavelength range. In particular, the second radiation source 17 emits light in the UV-C spectral range. The second radiation source 17 may comprise a plurality of emitter units. The emitter units of the second radiation source 17 may irradiate the fluid with UV light from different directions. For example, and as indicated in FIG. 1, the emitter units of the second radiation source 17 may be arranged annularly around the flow channel 11.

Furthermore, the flow reactor system 10 comprises a control unit 19. The control unit 19 is provided and configured to control the emission intensity of the first radiation source 15 and/or the second radiation source 17 depending on the at least one state variable determined by the measuring system 12. For this purpose, the control unit 19 is electrically connected to the measuring system 12. This means that the measuring system 12 provides measurement data and/or state variables to the control unit 19. Based on the measurement data and state variables, the control unit 19 can determine an emission intensity of the radiation sources 15, 17 with which sufficient sterilization of the fluid is ensured. The control unit 19 is further electrically connected to the first radiation source 15 and the second radiation source 17. In the exemplary embodiment shown, the control unit 19 is electrically connected to the radiation sources 15, 17 via respective driver units 25, 27. The control unit 19 controls a drive current for the emitter units comprised by the first radiation source 15 via the driver unit 25, so that the emitter units emit light with the required emission intensity. Further, the control unit 19 controls a drive current for the emitter units comprised by the second radiation source 17 via the driver unit 27 so that the emitter units emit UV light with the required emission intensity.

FIG. 2 shows a further exemplary embodiment of the flow reactor system 10. The exemplary embodiment according to FIG. 2 differs from the exemplary embodiment according to FIG. 1, among other things, in that the flow reactor system 10 comprises a further measuring system 22. The further measuring system 22 is arranged between the measuring system 12 and the first irradiation zone 14. This means that the further measuring system 22 is arranged after the measuring system 12 in the flow direction 100 of the fluid. The further measuring system 22 comprises a further light emitter 36 and a further visual sensor 38. The measuring system 12 and the further measuring system 22 may be of the same type. The further measuring system 22 is provided and configured to redetermine a state variable determined by the measuring system 12. Alternatively or additionally, the further measuring system 22 determines a further state variable that differs from the state variable determined by the measuring system 12. The further measuring system 22 is also electrically connected to the control unit 19. The further measuring system 19 transmits measurement data and/or state variables to the control unit 19. In this way, in addition to the measurement data transmitted by the measuring system 12, further measurement data is available to the control unit 19, on the basis of which a required emission intensity can be determined.

The exemplary embodiment according to FIG. 2 further comprises an analysis system 40. The analysis system 40 is electrically connected to the control unit 19. Alternatively, the analysis system 40 is directly electrically connected to the measuring system 12 and/or the further measuring system 22. The analysis system 40 analyzes the measurement data and state variables determined by the measuring system 12 and further measuring system 22 to determine eventual patterns in the data. Patterns may occur in a temporal and/or spatial progression of the state variable(s). By pattern detection, the prediction accuracy may be improved for state variables. The analysis system 40 provides pattern information to the control unit 19. The control unit 19 may consider the pattern information to determine the required emission intensity of the radiation sources 15, 17.

In FIG. 3, possible arrangement concepts for emitter units of the first and/or second radiation source 15, 17 are shown. In particular, due to their small dimensions, the emitter units of the first radiation source 15 can be arranged in different ways on or in the flow channel 11. The shown arrangement concepts according to (a) to (d) can be used alternatively or combined with each other. This means that the flow reactor system 10 does not necessarily comprise all of the emitter arrangements shown in FIG. 3.

In section (a) of the flow channel 11, two radiation sources 15 or emitter units are shown, which are arranged outside the flow channel 11. The flow channel 11 can, in particular, be configured as a pipe. As indicated by arrows, the emitter units emit light and couple it into the flow channel 11 substantially perpendicular to the flow direction 100. For example, the coupling of light may take place via transparent windows 50. For example, the UV transparent windows 50 may be made of quartz glass which are integrated into the pipe. The pipe or at least the pipe section can comprise polytetrafluoroethylene (PTFE) as a material. PTFE comprises a reflectance of up to 97% for UV-C radiation. Therefore, as indicated, the UV radiation emitted by the emitter units can be reflected by the wall of the pipe at a variety of reflection angles. The reflected radiation is still available for disinfection of the fluid.

In section (b) of the flow channel 11, an alternative arrangement of the emitter units is shown in which UV radiation is coupled into the flow channel at an angle that is not perpendicular to the flow direction 100. For example, the emitter units may be tilted toward the flow channel 11. For example, the angle π at which the emitter units are tilted may be between 30° and 60°. The light is coupled into the flow channel via transparent windows 50. As indicated, the emitter units may be arranged on different sides of the flow channel. For example, the emitter units may be arranged opposite each other.

In section (c) of the flow channel 11, another alternative arrangement of the emitter units is shown. Here, the emitter units are arranged in the flow channel 11 and are tilted with respect to the flow direction 100. The angle π at which the emitter units are tilted may be, for example, between 30° and 60°. As indicated, the emitter units may be inserted into chambers 52 within the flow channel 11 so that the emitter units are not in direct contact with the fluid. In the exemplary embodiment shown, the emitter units are spaced apart from each other in the flow direction 100 and are arranged on alternating sides of the flow channel 11. In this way, turbulence of the fluid can advantageously be achieved and disinfection by means of UV irradiation can be improved.

In section (d) of the flow channel 11 of FIG. 3, emitter units are inserted into a chamber 52 of the flow channel 11 in such a way that they couple radiation into the flow channel 11 in and against the flow direction 100 of the fluid. In the lower area of FIG. 3, a corresponding cross-section of the flow channel 11 can be seen. Due to the advantageous arrangement of the emitter units, an irradiation time can be extended compared to perpendicular light coupling.

FIG. 4 shows another exemplary embodiment of the flow reactor system 10. The flow reactor system 10 shown in FIG. 4 comprises a recirculation channel 60, which is provided and configured to recirculate a portion of the fluid at a position after the first 14 and second irradiation zones 16 in the flow direction 100 to a position before the irradiation zones 14, 16. This means that an inlet 64 of the recirculation channel 60 is arranged at an outlet-side position of the flow channel 11, while an outlet 66 of the recirculation channel 60 is arranged at an inlet-side position of the flow channel 11. The flow reactor system 10 further comprises valves 62 for opening and closing the inlet 64 and the outlet 66 of the recirculation channel 60. The valves 62 are configured as two-way valves. For example, the valves 62 are also controlled by the control unit 19 (not shown).

In the exemplary embodiment shown in FIG. 4, a further measuring system 22 is arranged after the irradiation zones 14, 16 in the flow direction 100. The further measuring system 22 can be electrically connected to the control unit 19 (not shown). The further measuring system 22 is provided and configured for determining at least one state variable after the UV irradiation. The state variable determined by the further measuring system 22 may be identical to or different from the state variable determined by the measuring system 12. For example, both the measuring system 12 and the further measuring system 22 determine a contamination level of the fluid. Alternatively, for example, the measuring system 12 determines a flow rate of the fluid while the further measuring system 22 determines, for example, a contamination level of the fluid. By means of a further measuring system 22 arranged after the irradiation zones 14, 16, the success of the irradiation with respect to the disinfection of the fluid can be determined. Furthermore, depending on the state variable determined by the further measuring system 22, it can be controlled whether and/or how much fluid is recirculated via the recirculation channel 60 in order to be subjected to a further irradiation in the irradiation zones 14, 16. The recirculation channel 60 of the exemplary embodiment shown in FIG. 4 comprises a buffer region 68 that is provided and configured to receive a portion of the fluid. By the buffer region 68, a portion of the fluid can be retained to later expose it to a repeated irradiation.

FIG. 5 shows an exemplary temporal progression of the emission intensity I. The variation of the emission intensity I can be achieved, for example, by adjusting the respective drive currents via the control unit 19 and the driver units 25, 27. In a first time range T₁, the required emission intensity I determined by the control unit 19 is below a maximum intensity 70 that can be achieved with LED-only irradiation, i.e. with the first radiation source 15. Accordingly, only the first LED-based radiation source 15 is controlled by the control unit 19, while the second radiation source 17 is deactivated. The determined emission intensity I can be proportional to the determined flow rate, the contamination level, or the turbidity level of the fluid. In a second time range T₂, the required emission intensity I determined by the control unit 19 is slightly above the maximum intensity 70 that can be achieved with LED-only illumination, i.e. with the first radiation source 15. In this scenario, both the first radiation source 15 and the second radiation source 17 are used to disinfect the fluid with UV radiation. However, since the threshold 70 is only slightly exceeded, it is sufficient to switch on only single lamp-based emitter units of the second radiation source, or to operate them dimmed. This means that at least one emitter unit of the second radiation source 17 is dimmed or switched off because a corresponding threshold value of at least one state variable is undershot.

In a third time range T₃, the required emission intensity I determined by the control unit 19 is significantly higher than the maximum intensity 70 that can be achieved with LED-only illumination, i.e. with the first radiation source 15. In this scenario, the second lamp-based radiation source can be operated at full nominal power to treat the fluid with the maximum available UV intensity.

In a fourth time range T₄, the required emission intensity I determined by the control unit 19 has again fallen below the maximum intensity 70 that can be achieved with LED-only illumination, i.e. with the first radiation source 15. Accordingly, the second radiation source 17 can be deactivated. The illumination with the LED-based first radiation source 15 is sufficient in this scenario.

In a fifth time range T₅, the state variable determined by the measuring system 12 is such that the irradiation dose can be further reduced while maintaining sufficient sterilization of the fluid. This means that the drive current of the first radiation source 15 can be reduced so that at least one LED-based emitter unit of the first radiation source 15 is switched off or operated in a dimmed manner. Accordingly, the emission intensity I decreases compared to the fourth time range T₄.

As indicated in FIG. 6, the flow reactor system 10 may be integrated in a processing plant 200. For example, the processing plant may be a processing plant for liquids, such as water. The processing plant may also be a processing plant for gases, such as air. For example, the processing plant may be a water processing plant or an air conditioning plant.

The features and exemplary embodiments described in connection with the figures can be combined with each other according to further exemplary embodiments, even if not all combinations are explicitly described. Furthermore, the exemplary embodiments described in connection with the figures may alternatively or additionally comprise further features according to the description in the general part.

The invention is not limited to the exemplary embodiments by the description based thereon. Rather, the invention encompasses any new feature as well as any combination of features, which in particular comprises any combination of features in the patent claims, even if this feature or combination itself is not explicitly stated in the patent claims or exemplary embodiments.

This patent application claims priority of patent application US 63/218,116, the content of which is hereby incorporated by reference.

### LIST OF REFERENCE SIGNS

10 flow reactor system
11 flow channel
12 measuring system
14 first irradiation zone
15 first radiation source
16 second irradiation zone
17 second radiation source
19 control unit
22 further measuring system
25, 27 driver unit
32, 36 light emitter
34, 38 light sensor
40 analysis system
50 transparent window
52 chamber
60 recirculation channel
62 valve
64 inlet of the recirculation channel
66 outlet of the recirculation channel
68 buffer region
70 maximum light intensity achievable with LEDs
100 flow direction
200 processing plant
I emission intensity
t time
T₁-T₅ time range
α angle

1. A flow reactor system for disinfecting a fluid, comprising:
a measuring system for determining at least one state variable,
a first irradiation zone and a second irradiation zone in which the fluid is irradiated with electromagnetic radiation,
a first radiation source arranged in the first irradiation zone, wherein the first radiation source comprises at least one emitter unit comprising a light-emitting diode which, during operation, emits light in a visible and/or ultraviolet wavelength range,
a second radiation source arranged in the second irradiation zone, wherein the second radiation source comprises at least one emitter unit comprising a lamp which, during operation, emits light in an ultraviolet wavelength range, and
a control unit which is provided and configured to control the emission intensity of the first radiation source and/or the second radiation source depending on the at least one state variable determined by the measuring system.

2. The flow reactor system according to claim 1, wherein the measuring system, the first irradiation zone and the second irradiation zone are arranged along a flow channel of the fluid.

3. The flow reactor system according to claim 1, wherein the second irradiation zone is arranged after the first irradiation zone in the flow direction of the fluid.

4. The flow reactor system according to claim 1, wherein the first irradiation zone and the second irradiation zone are arranged after the measuring system in the flow direction of the fluid.

5. The flow reactor system according to claim 1, wherein the state variable determined by the measuring system comprises at least one of the following group: a contamination level of the fluid, a flow rate of the fluid, a turbidity level of the fluid, a pressure of the fluid, a state variable independent of the fluid.

6. The flow reactor system according to claim 1, further comprising: a recirculation channel which is provided and configured to recirculate a portion of the fluid at a position which is located after the first and/or second irradiation zone in the flow direction to a position before the irradiation zones.

7. The flow reactor system according to claim 6, further comprising: valves for opening and closing an inlet and an outlet of the recirculation channel.

8. The flow reactor system according to claim 6, wherein the recirculation channel comprises a buffer region which is provided and configured to receive a portion of the fluid.

9. The flow reactor system according to claim 1, wherein the emitter units of the first and the second radiation source emit light in the UV-C wavelength range during operation.

10. The flow reactor system according to claim 1, wherein the first radiation source comprises at least one further emitter unit comprising a light emitting diode which, during operation, emits light in the UV-A and/or visible wavelength range.

11. The flow reactor system according to claim 1, further comprising: at least one further measuring system arranged after the measuring system in the flow direction of the fluid, which is provided and configured to redetermine the at least one state variable determined by the measuring system- or to determine a further state variable different from the state variable determined by the measuring system.

12. The flow reactor system according to claim 1, further comprising: an analysis system which is provided and configured to determine patterns in a temporal and/or spatial progression of the at least one state variable and to provide pattern information to the control unit.

13. The flow reactor system according to claim 1, wherein at least one emitter unit of the first radiation source emits light in a direction that is not perpendicular to the flow direction of the fluid.

14. A processing plant comprising the flow reactor system according to claim 1.

15. A method for disinfecting a fluid in a flow reactor system, comprising:
determining at least one state variable by a measuring system,
guidance of the fluid through a first irradiation zone and through a second irradiation zone in which the fluid is irradiated with electromagnetic radiation, wherein
a first radiation source arranged in the first irradiation zone comprises at least one emitter unit comprising a light-emitting diode, which, during operation, emits light in a visible and/or ultraviolet wavelength range, and wherein
a second radiation source arranged in the second irradiation zone comprises at least one emitter unit comprising a lamp which, during operation, emits light in an ultraviolet wavelength range, and
control of the emission intensity of the first radiation source and/or the second radiation source, depending on the at least one state variable determined by the measuring system.

16. The method according to claim 15, wherein the control of the emission intensity comprises: dimming or switching off at least one emitter unit of the first radiation source if a first threshold value of the at least one state variable is undershot.

17. The method according to claim 15, wherein the control of the emission intensity further comprises: dimming or switching off at least one emitter unit of the second radiation source if a second threshold value of the at least one state variable is undershot.

18. The method according to claim 15, further comprising: recirculating a portion of the fluid at a position which is located after the first and/or second irradiation zone in the flow direction to a position before the irradiation zones if a third threshold value of the at least one state variable is exceeded.

19. The method according to claim 15, wherein the control of the emission intensity comprises: activation of the second radiation source if a warning level of the at least one state variable is exceeded, and deactivation of the second radiation source if the warning level or a cut-off level of the state variable is undershot.
